(19) United States
(12) Patent Application Publication
(10) Pub. No.: US 2023/0330620 A1
(43) Pub. Date: Oct. 19, 2023

(54) OFF-GAS UTILIZATION IN ELECTRICALLY HEATED REFORMING PLANT

(71) Applicant: HALDOR TOPSØE A/S, Kgs. Lyngby (DK)

(72) Inventor: Peter Mølgaard MORTENSEN, Roskilde (DK)

(73) Assignee: TOPSOE A/S, Kgs. Lyngby (DK)

(21) Appl. No.: 18/006,184

(22) PCT Filed: Aug. 19, 2021
(86) PCT No.: PCT/EP2021/073057

§ 371 Date: Jan. 20, 2023

(30) Foreign Application Priority Data
Aug. 21, 2020 (EP) 20192075.8

(51) Int. Cl. B01J 19/24; B01J 19/00; C01B 3/48; C01B 3/50; C01C 1/04; C07C 1/04; C07C 29/48

(52) U.S. Cl. CPC B01J 19/245; B01J 19/0013; C01B 3/48; C01B 3/506; C01C 1/0417; C07C 1/041; C07C 29/48; B01J 2219/00135; C01B 2203/0233; C01B 2203/046; C01B 2203/047; C01B 2203/061; C01B 2203/062; C01B 2203/068; C01B 2203/085; C01B 2203/1241; C01B 2203/84

(57) ABSTRACT

A plant and a method are provided in which a first feed including hydrocarbons is subjected to electrical steam methane reforming (e-SMR) to generate a first syngas stream. An upgrading section receives the syngas stream and generates a first product stream and an off-gas stream from the syngas stream. A power generator receives at least a portion of the off-gas stream and/or a portion of said first product stream from the upgrading section and/or a portion of said first feed and generates a second electricity flow. At least a portion of the second electricity flow is arranged to provide at least a part of the first electricity flow to the e-SMR reactor.

### TECHNICAL FIELD

A plant and a method are provided in which a first feed comprising hydrocarbons is subjected to electrical steam methane reforming (e-SMR) to generate a first syngas stream. An upgrading section receives the syngas stream and generates a first product stream and an off-gas stream from the syngas stream. A power generator receives at least a portion of the off-gas stream and/or a portion of said first product stream from the upgrading section and/or a portion of said first feed and generates a second electricity flow. At least a portion of the second electricity flow is arranged to provide at least a part of the first electricity flow to the e-SMR reactor. This technology enables an electrically-powered chemical plant with varying levels of electricity import, which can therefore deal with fluctuations in the supply of renewable electricity.

### BACKGROUND

Production of bulk chemicals from synthesis gas, like methanol and hydrogen, is often performed at the expense of generating a high volume of off-gas. Typical reforming plants use a fired reformer, where off-gas has typically been used as fuel for generating the steam required to provide the synthesis gas itself. Also, the off-gas is used as fuel for the burners of the fired reformer.

Electrical heated steam reformers are known e.g. from Wismann et al, Science 2019: Vol. 364, Issue 6442, pp. 756-759, WO2019/228798, and WO2019/228795. If fired steam reforming units are replaced by an electrically heated reformer, fuel for heating the reforming process is no longer required. Accordingly, the volume of the off-gas required for heating purposes is severely reduced and excess off-gas production can be a problem.

The current technology aims to close overall mass and energy balances of a plant in which electrically-heated steam reforming takes place. In particular, the present technology aims to make use of excess off-gas which may be produced in such a plant. Additionally, the present technology aims to handle fluctuations in the supply of renewable electricity, and to establish an independent supply of syngas in a chemical plant in case of electricity shut-off from the electricity supply.

### SUMMARY

In the following, reference is made to embodiments of the invention. However, it should be understood that the invention is not limited to specific described embodiments. Instead, any combination of the following features and elements, whether related to different embodiments or not, is contemplated to implement and practice the invention.

A plant is provided, said plant comprising;
a first feed comprising hydrocarbons,
one or more co-reactant feeds
an electrical steam methane reforming (e-SMR) reactor, wherein the e-SMR reactor is arranged to be heated by a first electricity flow, and wherein the e-SMR reactor is arranged to receive at least a portion of said first feed comprising hydrocarbons and at least a portion of said one or more co-reactant feeds, and generate a first syngas stream,
an upgrading section arranged to receive a syngas stream and generate at least a first product stream and an off-gas stream from said syngas stream,
a power generator arranged to receive at least a portion of said off-gas stream and/or a portion of said first product stream from the upgrading section and generate a second electricity flow,
wherein at least a portion of said second electricity flow is arranged to provide at least a part of the first electricity flow to the e-SMR reactor.

A method for providing a product stream from a first feed comprising hydrocarbons is also provided, by means of the plant described herein.

A method for operating a plant as described herein is also provided, said method comprising the step of switching from a plant operation mode A to a plant operation mode B or vice-versa, as further described herein.

Further details of the technology are set out in the following description text, the dependent claims and the appended figures.

### LEGENDS TO THE FIGURES

FIGS. 1-7 show various plant layouts according to the invention, all of which comprise an e-SMR reactor, an upgrading section, a power generator, various gas feeds/streams and various electricity flows.

### DETAILED DISCLOSURE

The current technology describes a synergy between implementing a gas engine for converting excess off-gas into electricity, where the produced electricity can be used directly in the electrical reformer instead. This provides a solution for a balanced small scale chemical plant without unused process streams from the plant, thereby reducing by-product formation.

Part of the scope of this technology is an electrically-driven chemical plant with low, or zero electricity import, which can deal with fluctuations in the level of renewable electricity available. An electrically-driven syngas production plant has a very high demand of electricity to run an electrically heated steam methane reformer. The stable operation of such a plant will be vulnerable to fluctuations in electricity supply from external sources and in particular to breakdowns of electricity supply. The present invention has provided a possibility to run the plant solely by the use of electricity generated inside the plant. Thus, the present invention is based on the recognition that it is possible to generate the required high level of electricity for running the plant inside the plant itself firstly by adding a power generator to the plant and secondly by generating therein electricity using at least a portion of an excess off-gas stream of the plant and/or a portion of the hydrocarbon feed and/or a portion of the product stream.

In the following, all percentages are given as volume %, unless otherwise specified. The term “substantially pure” should be understood as meaning more than 80% pure, ideally more than 90%, such as more than 99% pure.

A plant is therefore provided as illustrated schematically in the Figures. In general terms, the plant comprises;
a first feed comprising hydrocarbons,
one or more co-reactant feeds
an electrical steam methane reforming (e-SMR) reactor,
an upgrading section, and
a power generator.

### First Feed

The first feed comprises hydrocarbons. In this context, the term “first feed comprising hydrocarbons” is meant to denote a gas with one or more hydrocarbons and possibly other constituents. Thus, first feed gas comprising hydrocarbons typically comprises a hydrocarbon gas, such as CH₄ and optionally also higher hydrocarbons often in relatively small amounts, in addition to small amounts of other gasses. Higher hydrocarbons are components with two or more carbon atoms such as ethane and propane. Examples of “first feed comprising hydrocarbons” may be natural gas, town gas, naphtha or a mixture of methane and higher hydrocarbons, biogas or LPG. Hydrocarbons may also be components with other atoms than carbon and hydrogen such as oxygen or sulphur.

The first feed may additionally comprise - or be mixed with one more co-reactant feeds -steam, hydrogen and possibly other constituents, such as carbon monoxide, carbon dioxide, nitrogen and argon. Typically, the first feed has a predetermined ratio of hydrocarbon, steam and hydrogen, and potentially also carbon dioxide.

In one aspect, the first feed is a biogas feed. Biogas is a mixture of gases produced by the breakdown of organic matter in the absence of oxygen. Biogas can be produced from raw materials such as agricultural waste, manure, municipal waste, plant material, sewage, green waste or food waste. Biogas is primarily methane (CH₄) and carbon dioxide (CO₂) and may have small amounts of hydrogen sulfide (H₂S), moisture, siloxanes, and possibly other components. Up to 30% or even 50% of the biogas may be carbon dioxide. The inherent mixture of CO₂ and CH₄ makes it a good feedstock for methanol production by e-SMR (“e-SMR-MeOH”), where essentially all carbon atoms can be converted into methanol.

When the first feed of hydrocarbons reaches the e-SMR reactor, it will have gone through at least steam addition (present as a co-reactant feed) and optionally also pretreatment (described in more detail in the following).

### Co-Reactant Feeds

The plant comprises one or more co-reactant feeds. The co-reactant feed(s) is/are suitably selected from a steam feed, a hydrogen feed, or a CO₂ feed. The co-reactant feeds are fed to the e-SMR reactor, preferably as a mixture with the first feed comprising hydrocarbons. The co-reactant feeds are among other aspects used to adjust the composition of the synthesis gas leaving the e-SMR according to thermodynamic considerations.

If the plant comprises a pre-treatment section, upstream the e-SMR reactor, co-reactant feeds can be added at different places in the pre-treatment section, e.g. hydrogen can be added upstream an hydrodesulfurization to facilitate hydrogenation reactions, and/or steam can be added upstream a prereformer to facilitate reforming reactions, and/or CO₂ can be added to a gas conditioning unit to partly shift the feed gas according to the water-gas shift reaction.

### e-SMR Reactor

The plant comprises an electrical steam methane reforming (e-SMR) reactor. The e-SMR reactor performs the steam methane reforming reaction on the first feed and any co-reactant feeds.

The e-SMR reactor is arranged to receive at least a portion of said first feed comprising hydrocarbons and at least a portion of said one or more co-reactant feeds and generate a first syngas stream from said first feed (mixed with the co-reactant feed(s)).

The term “steam reforming” or “steam methane reforming reaction” is meant to denote a reforming reaction according to one or more of the following reactions:

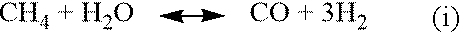

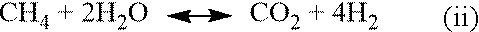

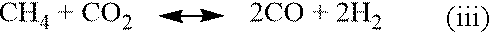

Reactions (i) and (ii) are steam methane reforming reactions, whilst reaction (iii) is the dry methane reforming reaction.

For higher hydrocarbons, viz. CₙHₘ, where n≥2, m ≥ 4, equation (i) is generalized as:

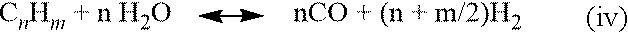

where n≥2, m ≥ 4.

Typically, steam reforming is accompanied by the water gas shift reaction (v):

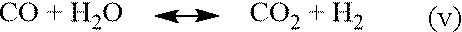

The terms “steam methane reforming” and “steam methane reforming reaction” are meant to cover the reactions (i) and (ii), the term “steam reforming” is meant to cover the reactions (i), (ii) and (iv), whilst the term “methanation” covers the reverse reaction of reaction (i). In most cases, all of these reactions (i)-(v) are at, or close to, equilibrium at the outlet from the reforming reactor.

Since the electrically heated reforming reactor is electrically heated, less overall energy consumption takes place compared to a fired steam methane reforming reactor, since a high temperature flue gas of the reforming reactor is avoided. Moreover, if the electricity utilized for heating the electrically heated reforming reactor and possibly other units of the synthesis gas plant is provided from renewable energy resources, the overall consumption of hydrocarbons for the synthesis gas plant is minimized and CO₂ emissions accordingly reduced.

The e-SMR reactor is arranged to be heated by a first electricity flow. In an embodiment, the electrically heated reforming reactor of the synthesis gas plant comprises:
a pressure shell housing an electrical heating unit arranged to heat the first catalyst, where the first catalyst comprises a catalytically active material operable to catalyzing steam reforming of the first part of the feed gas, wherein the pressure shell has a design pressure of between 5 and 50 bar,
a heat insulation layer adjacent to at least part of the inside of the pressure shell, and at least two conductors electrically connected to the electrical heating unit and to an electrical power supply placed outside the pressure shell,
wherein the electrical power supply is dimensioned to heat at least part of the first catalyst to a temperature of at least 500° C. by passing an electrical current through the electrical heating unit.

An important feature of the electrically heated reforming reactor is that the energy is supplied inside the reforming reactor, instead of being supplied from an external heat source via heat conduction, convection and radiation, e.g. through catalyst tubes. In an electrically heated reforming reactor with an electrical heating unit connected to an electrical power supply via conductors, the heat for the reforming reaction is provided by resistance heating. The hottest part of the electrically heated reforming reactor will be within the pressure shell of the electrically heated reforming reactor. Preferably, the electrical power supply and the electrical heating unit within the pressure shell are dimensioned so that at least part of the electrical heating unit reaches a temperature of 850° C., preferably 900° C., more preferably 1000° C. or even more preferably 1100° C.

In an embodiment, the electrically heated reformer comprises a first catalyst as a bed of catalyst particles, e.g. pellets, typically in the form of catalytically active material supported on a high area support with electrically conductive structures embedded in the bed of catalyst particles. Alternatively, the catalyst may be catalytically active material supported on a macroscopic structure, such as a monolith.

When the electrically heated reforming reactor comprises a heat insulation layer adjacent to at least part of the inside of the pressure shell, appropriate heat and electrical insulation between the electrical heating unit and the pressure shell is obtained. Typically, the heat insulation layer will be present at the majority of the inside of the pressure shell to provide thermal insulation between the pressure shell and the electrical heating unit/first catalyst; however, passages in the heat insulation layers are needed in order to provide for connection of conductors between the electrical heating unit and the electrical power supply and to provide for inlets/outlets for gasses into/out of the electrically heated reforming reactor.

The presence of heat insulating layer between the pressure shell and the electrical heating unit assists in avoiding excessive heating of the pressure shell, and assists in reducing thermal losses to the surroundings of the electrically heated reforming reactor. The temperatures of the electrical heating unit may reach up to about 1300° C., at least at some parts thereof, but by using the heat insulation layer between the electrical heating unit and the pressure shell, the temperature of the pressure shell can be kept at significantly lower temperatures of e.g. 500° C. or even 200° C. This is advantageous since typical construction steel materials are unsuitable for pressure bearing applications at high temperatures, such as above 1000° C. Moreover, a heat insulating layer between the pressure shell and the electrical heating unit assists in control of the electrical current within the reforming reactor, since heat insulation layer is also electrically insulating. The heat insulation layer could be one or more layers of solid material, such as ceramics, inert material, refractory material or a gas barrier or a combination thereof. Thus, it is also conceivable that a purge gas or a confined gas constitutes or forms part of the heat insulation layer.

As the hottest part of the electrically heated reforming reactor during operation is the electrical heating unit, which will be surrounded by heat insulation layer, the temperature of the pressure shell can be kept significantly lower than the maximum process temperature. This allows for having a relative low design temperature of the pressure shell of e.g. 700° C. or 500° C. or preferably 300° C. or 200° C. of the pressure shell whilst having maximum process temperatures of 800° C. or 900° C. or even 1100° C. or even up to 1300° C.

Another advantage is that the lower design temperature compared to a fired SMR means that in some cases the thickness of the pressure shell can be decreased, thereby saving costs.

It should be noted that the term “heat insulating material” is meant to denote materials having a thermal conductivity of about 10 W·m⁻¹·K⁻¹ or below. Examples of heat insulating materials are ceramics, refractory material, alumina-based materials, zirconia- based materials and similar.

Further details of the e-SMR are laid out in Wismann et al, (2019) “Electrified methane reforming: A compact approach to greener industrial hydrogen production” Science Vol. 364, Issue 6442, pp. 756-759, the contents of which are incorporated by reference.

It is conceivable that the e-SMR is placed in parallel or series to an SMR, an autothermal reformer (ATR), and/or heat exchange reformer (HTER). Such arrangements are described in co-pending applications PCT/EP2020/055173, PCT/EP2020/055174 and PCT/EP2020/055178 which are hereby incorporated by reference. In an embodiment, the e-SMR could work in parallel/series to an ATR, SMR, and/or HTER, to generate a first syngas stream as per this invention.

### Upgrading Section

The plant comprises an upgrading section arranged to receive a syngas stream and generate at least a first product stream and an off-gas stream from said syngas stream. The first product stream may e.g. be a hydrogen gas, a carbon monoxide gas, higher hydrocarbons, synthetic fuels, methanol, or ammonia.

The syngas stream supplied to the upgrading section may be the syngas stream generated in the e-SMR. Therefore, the upgrading section may be arranged to receive the syngas stream (and suitably the entire syngas stream) generated by the e-SMR reactor.

Hydrogen and methanol upgrading sections are preferred because — in their classical configurations — they have a large by-product of off-gas.

In one preferred aspect,
the upgrading section is a hydrogen purification section,
the first product stream is a hydrogen-rich stream, and
the off-gas stream is an off-gas stream from the hydrogen purification section.

In an embodiment, the hydrogen purification section may be a swing adsorption unit, such as pressure swing adsorption (PSA unit) or temperature swing adsorption (TSA unit). The off-gas stream from the hydrogen purification section may comprise CH₄, CO₂, H₂, N₂, and CO.

By swing adsorption, a unit for adsorbing selected compounds is meant. In this type of equipment, a dynamic equilibrium between adsorption and desorption of gas molecules over an adsorption material is established. The adsorption of the gas molecules can be caused by steric, kinetic, or equilibrium effects. The exact mechanism will be determined by the used adsorbent and the equilibrium saturation will be dependent on temperature and pressure. Typically, the adsorbent material is treated in the mixed gas until near saturation of the heaviest compounds and will subsequently need regeneration. The regeneration can be done by changing pressure or temperature. In practice, this means that a process with at least two units is used, saturating the adsorbent at high pressure or low temperature initially in one unit, and then switching unit, now desorbing the adsorbed molecules from the same unit by decreasing the pressure or increasing the temperature. When the unit operates with changing pressures, it is called a pressure swing adsorption unit, and when the unit operates with changing temperature, it is called a temperature swing adsorption unit. Pressure swing adsorption can generate a hydrogen purity of 99.9% or above.

In a further aspect, also being preferred,
the upgrading section is a methanol synthesis section,
the first product stream is a methanol-rich stream, and
the off-gas stream is an off-gas stream from methanol synthesis section.

The methanol synthesis section may be as described in J.B. Hansen, P.E.H. Nielsen, Methanol Synthesis, Handbook of heterogeneous catalysis, John Wiley & Sons, Inc., New York, 2008, pp. 2920-2949. The off-gas stream from the methanol synthesis section may comprise CO, H₂, CO₂, CH₃OH, CH₄, and N₂.

In another aspect,
the upgrading section is a CO cold box,
the upgrading section being arranged to receive a syngas stream and generate a first product stream being a substantially pure CO stream, a second product stream, being a substantially pure H₂ stream and an off-gas stream from the CO cold box.

The CO cold box may be as described in “Carbon Monoxide” in Kirk-Othmer Encyclopedia of Chemical Technology ECT (online), 2000, by Ronald Pierantozzi. The off-gas stream from the CO cold box may comprise CH₄, CO, H₂, and N2.

In yet a further aspect,
the upgrading section is an ammonia loop,
the product stream is a substantially pure ammonia stream, and
the off-gas stream is an off-gas stream from the ammonia loop.

The ammonia loop may be as described in I. Dybkjær, Ammonia production processes, in: A. Nielsen (Ed.) Ammonia - catalysis and manufacture, Springer, Berlin, Germany, 1995, pp. 199-328. The off-gas stream from the ammonia loop may comprise NH₃, H₂, CH₄, and N₂.

In a further aspect,
the upgrading section is a Fischer-Tropsch section,
the product stream is a stream of higher hydrocarbons, and
the off-gas stream is an off-gas stream from the Fischer-Tropsch section.

The Fischer-Tropsch section may be as described in Dry, M.E. (2008). The Fischer-Tropsch(FT) Synthesis Processes. In Handbook of Heterogeneous Catalysis (eds. G. Ertl, H. Knözinger, F. Schüth and J. Weitkamp), 2008. The off-gas stream from the Fischer-Tropsch section may comprise hydrocarbons (as ethane, ethene, propene, and propane), CH₄, H₂, CO, and N₂. In this aspect, the term “higher hydrocarbons” is understood as meaning condensable hydrocarbons, such as hexane, heptane, heptene, octane, etc.

In one aspect of the plant, the upgrading section is arranged to receive the syngas stream generated by the e-SMR reactor; i.e. directly without a change in the syngas composition.

It may also be possible that the syngas stream generated by the e-SMR reactor is passed through one or more additional reactors or units before it reaches the upgrading section (see below).

### Power Generator

The present technology is based on the realisation that fuel rich off-gas streams rarely have any commercial value. However, they are often combustible, and can therefore be used accordingly in the plant itself.

The plant therefore comprises a power generator arranged to receive at least a portion of (and preferably all of) said off-gas stream and/or a portion of said first product stream from the upgrading section and generate a second electricity flow.

Preferably, the power generator is arranged to receive at least a portion of the off-gas stream from the upgrading section and generate a second electricity flow. This arrangement optimises the use of off-gas streams in the plant.

In addition to the off-gas stream and/or the first product stream, an external fuel may also be imported to drive the power generator, i.e. an import fuel. Import fuel can be obtained as a by-product from another chemical plant, or natural gas, biogas, or similar.

The power generator may also be arranged to receive a portion of the first feed comprising hydrocarbons and generate the second electricity flow. The mixed feed with steam etc. is not fed to the power generator.

As mentioned, a power generator provides electrical power from a combustible gas stream. Various arrangements of power generators may be known to the skilled person. A suitable power generator may be a gen-set in which a first module (e.g. an internal combustion engine) converts combustible gas into mechanical energy (e.g. rotational energy). A second module (e.g. a generator) is coupled to the first module, so as to convert the mechanical energy into electrical power. A fuel cell, such as a hydrogen fuel cell, can also be used as a power generator. A specific example of a power generator is a Combined Heat and Power (CHP) unit. Another example of a power generator is a gas turbine.

It is conceivable that a gas storage is included in the plant to allow collection of the off-gases during high production periods, and in this way even out the operation of the power generator, and even sometimes have a stop-start scenario for this unit. This comes down to practical operation of the unit, which in some cases can become to inefficient when operating with too low fuel.

The skilled person will be able to select the particular power generator and the operating parameters thereof, depending on e.g. the particular gas stream input available and the desired electricity flow output.

### Electricity Flows

At least a portion of, and preferably the entirety of, the second electricity flow (from the power generator) is arranged to provide at least a part of, and preferably the entirety of, the first electricity flow to the e-SMR reactor.

In this manner, effective use of the off-gas stream and/or the first product stream is possible. Additionally, the configuration gives an improved agility in operation. In one particular aspect, when renewable electricity is used for chemical production, a central problem is security of electricity supply, and this invention allows for a continued operation despite electricity interruption.

An electricity supply unit may be arranged to receive the second electricity flow from the power generator, and optionally the external electricity flow, and provide the first electricity flow to the e-SMR reactor. The electricity supply unit allows the relative proportions of second electricity flow and external electricity flow to be balanced, according to the availability of each electricity flow, in particular when the external electricity flow is provided by a source of renewable electricity.

In one aspect, an external electricity flow may be arranged to provide part of the first electricity flow to the e-SMR reactor. This external electricity flow may thus supplement the second electricity flow to the e-SMR reactor, e.g. in cases where electricity generation in the second electricity flow is not sufficient to drive the e-SMR reactor.

In one useful aspect, a source of renewable electricity is arranged to provide said external electricity flow. This not only reduces the environmental impact of the present invention, but also allows the second electricity flow (from the power generator) to be used to compensate for variations in the external electricity flow from the renewable source.

The second electricity flow may constitute the entire first electricity flow required to heat the e-SMR reactor. An external electricity flow may therefore be avoided, thus reducing the overall electricity requirements of the plant.

Optionally, the second electricity flow generated by the power generator is larger than the first electricity flow. In this manner, the external electricity flow can be avoided, plus the plant can export electricity for other uses, external to the plant or to other electricity driven utilities in the plant, such as compressors and pumps.

### Additional Reactors

As noted, the plant may comprise one or more additional reactors or units arranged between the e-SMR reactor and the upgrading section. Typically, these additional reactors or units are arranged to adjust the content of the syngas, so that it is best suited to the particular upgrading section in which it is to be used.

In one aspect, the plant further comprises at least one water gas shift (WGS) reactor arranged downstream the e-SMR reactor. The at least one WGS reactor is arranged to receive at least a portion of the first syngas stream from the e-SMR reactor and generate a second syngas stream from said first syngas stream. At least a portion of said second syngas stream is then fed to the upgrading section. Two WGS reactors are very commonly used, placed in series with interstage cooling. Also three WGS reactors in series are conceivable.

The plant may further comprise one or more gas conditioning units arranged between the e-SMR reactor and the upgrading section. These one or more gas conditioning units may be selected from: a flash separation unit, a CO₂ removal section, a methanator, or a combination of such units.

In addition, heat exchangers may be included in the plant layout, as required for temperature control and energy optimization. Also steam generators (boilers) can be used accordingly.

The plant may comprise a pre-treatment section upstream the e-SMR reactor. The pre-treatment section is arranged to pre-treat the first feed of hydrocarbons before it is fed to the e-SMR reactor. The pre-treatment section typically comprises one or more pre-treatment units selected from a gas adjustment unit, a heating unit, a hydrodesulfurisation (HDS) unit and a pre-reforming unit.

By “gas adjustment unit” is understood a unit operation for adjusting the composition of the gas. Examples of such units could be: a polymer membrane, a ceramic membrane, a pressure swing adsorption (PSA) unit, or a temperature swing adsorption (TSA) unit. The gas adjustment unit can be used for partially removing undesired component in feed gas. As examples, a membrane can be used to partly remove CO₂ from a hydrocarbon containing gas and a PSA can be used to remove higher hydrocarbons from a hydrocarbon containing gas.

In the case where the pre-treatment section comprises a heating unit, a portion of the off-gas stream from the upgrading section may be arranged to be returned to the pre-treatment section and used as fuel for said heating unit. This allows the amount of external fuel used for heating to be reduced, and can help optimise the use of the off-gas stream.

### Methods

The present technology also provides a method in which the above-described plant is utilized.

A method for providing a product stream from a first feed comprising hydrocarbons is thus provided. The method comprises the steps of:
providing a plant according to any one of the preceding claims,
feeding at least a portion of the first feed comprising hydrocarbons to the electrical steam methane reforming (e-SMR) reactor, and heating said e-SMR reactor with a first electricity flow so as to generate a syngas stream from said first feed,
feeding syngas stream to the upgrading section and generating at least a product stream and an off-gas stream from said syngas stream,
feeding at least a portion of said off-gas stream and/or a portion of said first product stream from the upgrading section and/or a portion of said first feed to the power generator and generating a second electricity flow,
feeding at least a portion of said second electricity flow to provide at least a part of the first electricity flow to the e-SMR reactor.

All details provided for the plant of the invention, above, are equally relevant for the method of the invention, mutatis mutandis.

The current technology allows the plant to deal with fluctuations in the level of electricity available. This is specifically an important aspect when the external electricity is provided from renewable electricity sources with high fluctuations. A method for operating a plant is thus described, wherein;
in a first plant operation mode A, the first electricity flow to the e-SMR reactor, comprises a first proportion (A1) of the second electricity flow and a first proportion (A2) of the external electricity flow;
in a second plant operation mode B, the first electricity flow to the e-SMR reactor, comprises a second proportion (B1) of the second electricity flow and a second proportion (B2) of the external electricity flow;
wherein the first proportion (A1) of the second electricity flow in the first plant operation mode A is smaller than the second proportion (B1) of the second electricity flow in the second plant operation mode B;
and wherein the first proportion (A2) of the external electricity flow in the first plant operation mode A is larger than the second proportion (B2) of the external electricity flow in the second plant operation mode B;
said method comprising the step of switching from plant operation mode A to plant operation mode B or vice-versa.

In the second plant operation mode B - the second proportion (B1) of the second electricity flow in the first electricity flow may be 75% or more, 80% or more, 90% or more, or 100%. In the second plant operation mode B, the first electricity flow to the e-SMR reactor may consist of the second electricity flow; and the second proportion (B2) of the external electricity flow is zero. In other words, in these aspects, the second electricity flow from the power generator makes up most, or even all, of the first electricity flow.

In one aspect of this method, the first electricity flow in the second plant operation mode B is lower than the first electricity flow in the first plant operation mode A.

The step of switching from plant operation mode A to plant operation mode B may at least partially obtained by increasing off-gas production in the upgrading section. Increased off-gas production leads to increased second electricity flow, which can reduce the proportion of external electricity flow required.

The step of switching from plant operation mode A to plant operation mode B may at least partially be obtained by feeding part of the first feed directly to the power generator.

The step of switching from plant operation mode A to plant operation mode B may also at least partially be obtained by decreasing said first electricity flow.

In the case where the external electricity flow is provided from a renewable source of electricity, the step of switching from plant operation mode A to plant operation mode B may take place when the external electricity flow available from said renewable source of electricity drops below a predetermined level. Also, when the external electricity flow is provided from a renewable source of electricity, the step of switching from plant operation mode B to plant operation mode A may take place when the external electricity flow available from said renewable source of electricity rises above a predetermined level. Again, such arrangements allow the plant to react to variations in the amount of renewable energy available.

The switch between operation mode A and B, or vice versa, typically takes place within a time period of 2 hours, more preferably within 1 hour, and most preferably within 0.5 hours after a preceding switch. This corresponds to the time period for which variations in renewable energy sources (e.g. wind power or solar power) can be accurately predicted.

### Detailed Description of the Figures

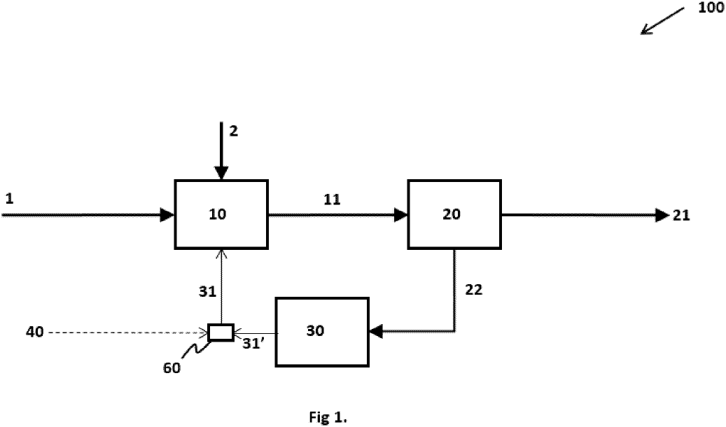

FIG. 1 illustrates a layout of a plant 100. A first feed 1 comprising hydrocarbons, and one or more co-reactant feeds 2 are fed to an electrical steam methane reforming (e-SMR) reactor 10. The e-SMR reactor 10 is arranged to be heated by a first electricity flow 31. The e-SMR reactor is arranged to receive at least a portion of the first feed 1 and at least a portion of the co-reactant feed 2. In turn, a first syngas stream 11 is generated in the e-SMR reactor 10, from the first feed 1 and the co-reactant feed(s) 2.

An upgrading section 20 is arranged to receive the syngas stream 11. The upgrading section 20 generates at least a first product stream 21 and an off-gas stream 22 from the syngas stream 11, 13a.

A power generator 30 is arranged to receive (in this embodiment, the entirety of) the off-gas stream 22 from the upgrading section 20 and generate a second electricity flow 31′.

The second electricity flow 31′ is provided from the power generator 30, to the electricity supply unit 60. The (optional) external electricity flow 40 is also provided to the electricity supply unit 60. The electricity supply unit 60 then provides first electricity flow 31 to the e-SMR reactor 10.

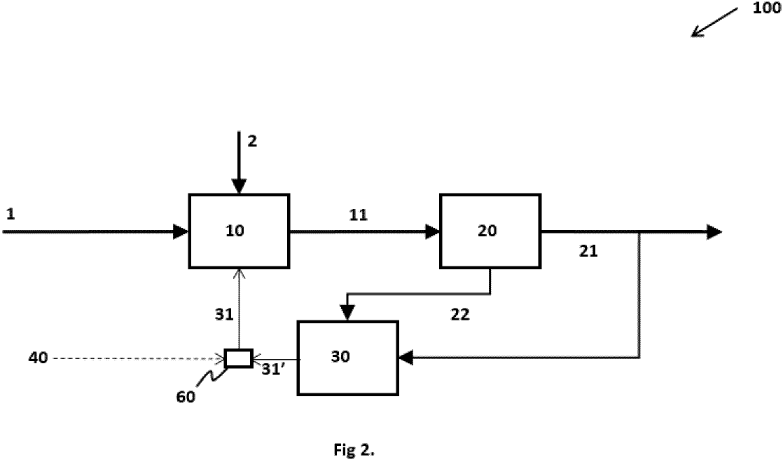

The layout of FIG. 2 is similar to that of FIG. 1. In FIG. 2, a portion of the first product stream 21 is also provided to the power generator 30, and used to generate second electricity flow 31′. This embodiment is advantageously used when the external electricity flow is insufficient for the operation of the e-SMR.

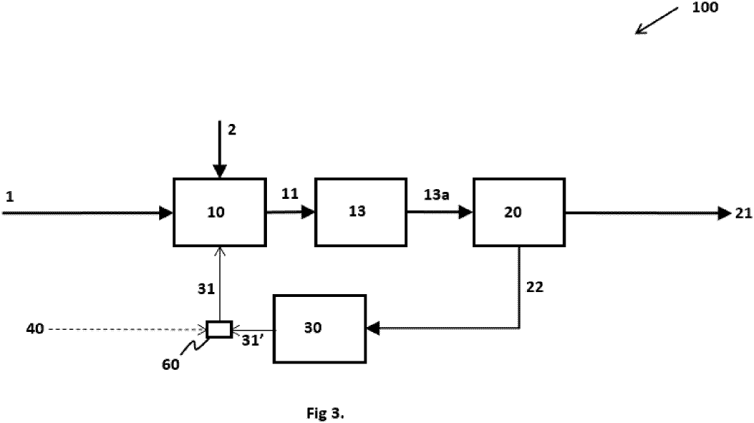

The layout of FIG. 3 is similar to that of FIG. 1. In FIG. 3, a water gas shift reactor 13 is arranged downstream the e-SMR reactor 10. The WGS reactor 13 is arranged to receive the first syngas stream 11 from the e-SMR reactor 10 and generate a second syngas stream 13a from said first syngas stream 11, typically being richer in hydrogen than the first syngas stream 11. As shown, at least a portion of the second syngas stream 13a is fed to the upgrading section 20.

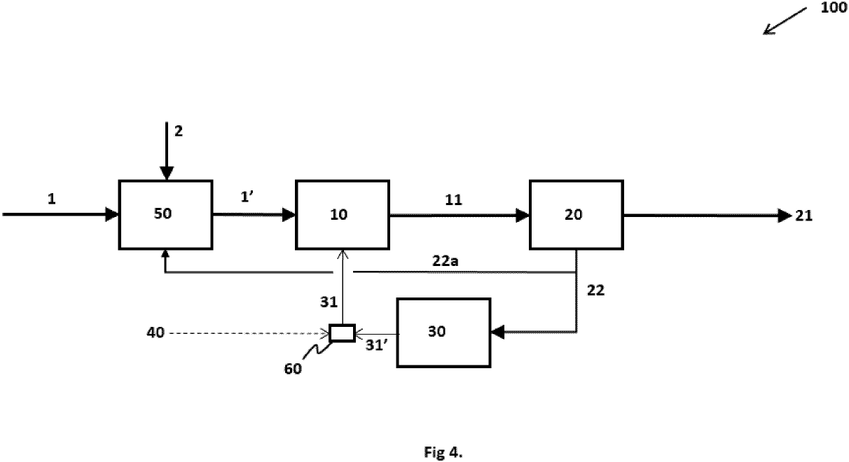

The layout of FIG. 4 is similar to that of FIG. 1. In FIG. 4, a pre-treatment section 50 in the form of a heating unit is arranged upstream the e-SMR reactor 10, and pre-treats the first feed 1 of hydrocarbons in combination with one or more of the co-reactant feeds 2 before the first feed 1 is fed to the e-SMR reactor 10. Also in this layout, a portion 22a of the off-gas stream 22 from the upgrading section 20 is fed to the pre-treatment section 50 and used as fuel for heating said pre-treatment section 50. This embodiment can be used to optimize the overall energy efficiency of the plant by utilizing the off-gas stream for preheating purpose, and having the electricity supply unit as balancing unit. In such a configuration, process control of the preheating can be achieved by regulating on the amount of fuel going to the electricity supply unit.

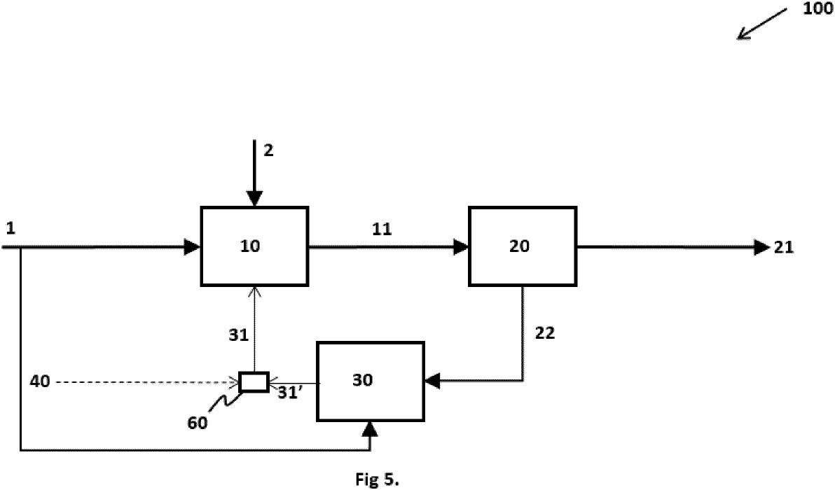

The layout of FIG. 5 is similar to that of FIG. 1. In FIG. 5, a portion of the first feed 1 is provided to the power generator 30 and contributes to generation of the second electricity flow 31′. This embodiment is advantageously used when the external electricity flow is insufficient for operation of the e-SMR.

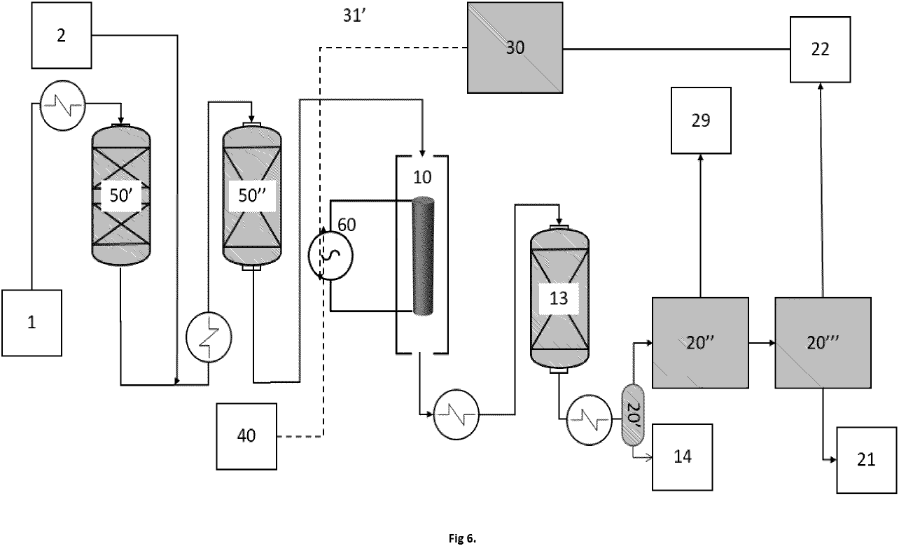

FIG. 6 illustrates a more detailed embodiment of a hydrogen plant. A first feed 1 comprising hydrocarbons, and also some hydrogen, is preheated initially and sent to a first pre-treatment step 50′ of hydrogenation and sulfur adsorption. A co-reactant feeds 2 comprising primarily steam is mixed into the effluent, and the combined gas is heated before entering a second pre-treatment step 50″ facilitating pre-reforming of higher hydrocarbons in the gas. The effluent is transferred to e-SMR reactor 10. This elevates the temperature and converts it - according to steam reforming and water gas shift equilibriums - to a synthesis gas comprising CO, H₂, CO₂, H₂O and CH₄. Outlet temperatures from this step can be 800° C., preferably 950° C., and even more preferably 1100° C. The synthesis gas is cooled to around 300-500° C. and sent to a water-gas-shift reactor 13, where CO reacts with H₂O to produce more H₂ and CO₂. The effluent is cooled to below the dew-point of the syngas stream 13a. The condensate of primarily liquid H₂O 14 is separated from the dry syngas in a separator 20′. The dry syngas is further upgraded in a CO₂ removal unit 20″, such as an amine wash, where the principal part of the CO₂ is removed as a by-product 29. The last upgrading step comprises a PSA, where the product is separated in to a hydrogen rich product 21 and an off-gas 22.

A power generator 30 is arranged to receive (in this embodiment, the entirety of) the off-gas stream 22 from the PSA 20‴ and generate a second electricity flow 31′. The second electricity flow 31′ is provided from the power generator 30, to the electricity supply unit 60. An external electricity flow 40 is also provided to the electricity supply unit 60. The electricity supply unit 60 then provides first electricity flow 31 to the e-SMR reactor 10.

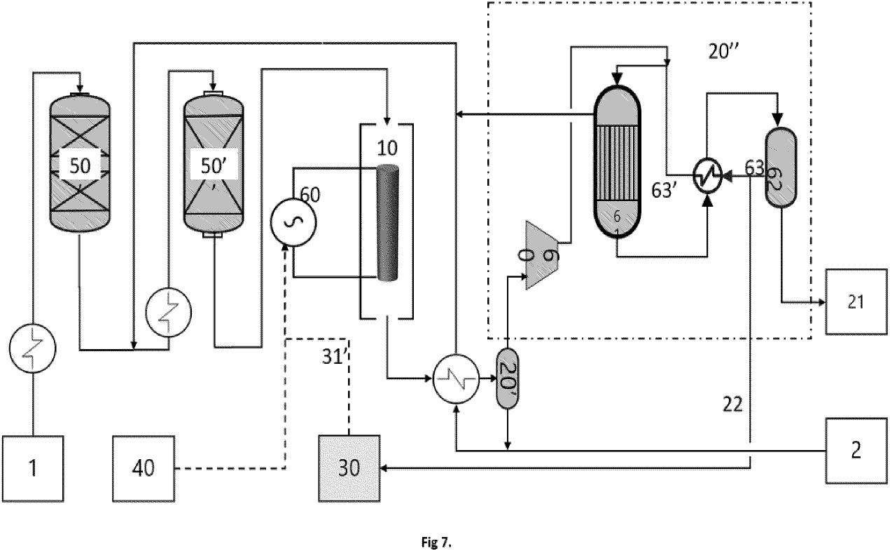

FIG. 7 illustrates a more detailed embodiment of a methanol plant. A first feed 1 comprising hydrocarbons, and also some hydrogen, and ideally also carbon dioxide, is preheated initially and sent to a first pre-treatment step 50′ of hydrogenation and sulfur adsorption. Co-reactant feeds 2 comprising primarily steam, are mixed into the effluent, and the combined gas is heated before entering a second pre-treatment step 50″ facilitating pre-reforming of higher hydrocarbons in the gas. The effluent is transferred to e-SMR reactor 10. This elevates the temperature and converts it — according to steam reforming and water gas shift equilibriums — to a synthesis gas comprising CO, H₂, CO₂, H₂O and CH₄. Outlet temperatures from this step can be 800° C., preferably 950° C., and even more preferably 1100° C. The effluent is cooled to below the dew-point of the syngas stream 11. The condensate of primarily liquid H₂O is separated from the dry syngas in a separator 20′. The dry syngas is further upgraded in a methanol synthesis unit 20″. In this embodiment, the methanol loop comprises a make-up gas compressor 60, a boiling water methanol reactor 61, and methanol flash separator 62, and an internal recycle of unconverted gas 63. Part of the unconverted gas 63′ is recycled to the boiling water methanol reactor 61, typically by a recycle compressor (not shown), while another part of the flow is purged from the loop as an off-gas 22.

A power generator 30 is arranged to receive (in this embodiment, the entirety of) the off-gas stream 22 from the methanol synthesis 20″ and generate a second electricity flow 31′. The second electricity flow 31′ is provided from the power generator 30, to the electricity supply unit 60. An external electricity flow 40 is also provided to the electricity supply unit 60. The electricity supply unit 60 then provides first electricity flow 31 to the e-SMR reactor 10.

### Example 1

Example 1 shows a methanol plant operating with a given feedstock (1) primarily of CH₄ and CO₂. This is mixed with steam as a co-reactant stream (2) and then reformed in an e-SMR to produce a synthesis gas product. When operating at an energy efficiency of 90%, the e-SMR uses 2790 kW as first electricity flow (31) in the given example. The synthesis gas goes through an upgrading section, including steps for temperature control and condensate removal from the synthesis gas from the e-SMR (10). In the upgrading section, the synthesis gas is compressed and mixed with a recycle stream, before reacting in a methanol reactor to produce methanol. Liquid methanol is condensed from this stream and the remaining gas is divided into one stream sent to a compressor making up the recycle stream to the methanol reactor. The remaining stream constitutes an off-gas stream which is transfer to the power generator (30) for generation of the second electricity flow (31′). The off-gas is this case has a LHV value of 3482 kcal/Nm³. Using a power generator with an electricity conversion efficiency of 48%, the size of the second electricity flow (31′) will be 1088 kW. During operation an external electricity flow (40) of additionally 1702 kW is provided to the e-SMR.

Example 1

| | Feed (1) | Add. Feed (2) | Inlet 50″ | Outlet 50″ | Inlet 10 | Outlet 10 | Inlet flash | Outlet flash | Outlet comp |
|---|---|---|---|---|---|---|---|---|---|
| T [°C] | 380 | 250 | 304 | 450 | 419 | 950 | 40 | 40 | 218 |
| P [barg] | 28.4 | 29.4 | 28.4 | 27.4 | 26.4 | 25.0 | 23.0 | 23.0 | 88.3 |
| Components [Nm³/h] | | | | | | | | | |
| Carbon Dioxide | 247 | 0 | 247 | 247 | 263 | 158 | 158 | 158 | 158 |
| Nitrogen | 5 | 0 | 5 | 5 | 5 | 5 | 5 | 5 | 5 |
| Methane | 741 | 0 | 741 | 741 | 738 | 126 | 126 | 126 | 126 |
| Ethane | 7 | 0 | 7 | 7 | 0 | 0 | 0 | 0 | 0 |
| Hydrogen | 16 | 0 | 16 | 16 | 78 | 1808 | 1808 | 1808 | 1808 |
| Carbon Monoxide | 0 | 0 | 0 | 0 | 2 | 718 | 718 | 718 | 718 |
| Water | 0 | 1133 | 1133 | 1133 | 110 0 | 593 | 593 | 9 | 9 |
| Oxygen | 0 | 0 | 0 | 0 | 0 | 0 | 0 | 0 | 0 |
| Methanol | 0 | 0 | 0 | 0 | 0 | 0 | 0 | 0 | 0 |

Example 1 continued

| | Add rec. | Inlet MeOH | Outlet MeOH | Inlet flash | Off-gas (22) | Recycle | Product (21) |
|---|---|---|---|---|---|---|---|
| T [°C] | 136 | 220 | 250 | 40 | 40 | 40 | 40 |
| P [barg] | 88.3 | 87.8 | 87.3 | 86.3 | 86.3 | 86.3 | 86.3 |
| Components [Nm³/h] | | | | | | | |
| Carbon Dioxide | 478 | 478 | 437 | 437 | 80 | 320 | 37 |
| Nitrogen | 24 | 24 | 24 | 24 | 5 | 19 | 0 |
| Methane | 594 | 594 | 594 | 594 | 117 | 468 | 9 |
| Ethane | 0 | 0 | 0 | 0 | 0 | 0 | 0 |
| Hydrogen | 3086 | 3086 | 1603 | 1603 | 320 | 1278 | 6 |
| Carbon Monoxide | 864 | 864 | 183 | 183 | 36 | 145 | 1 |
| Water | 9 | 9 | 50 | 50 | 0 | 0 | 49 |
| Oxygen | 0 | 0 | 0 | 0 | 0 | 0 | 0 |
| Methanol | 9 | 9 | 730 | 730 | 2 | 9 | 719 |

### Example 2

In another example, consider the same plant and process as presented in Example 1 with the same flow of feedstock and similar operation of the e-SMR. However in this case the plant has switched to a second plant operation mode B wherein les external electricity flow (40) is available. This case is summarized in Table 2. In this case the ratio between the recycle and the off-gas is switched, now sending 60% of the gas to the power generator (30), instead of 20% in Example 1. Consequently, the side of the off-gas stream increases. With a heating value of 3090 kcal/Nm³ and the same electricity conversion efficiency of 48%, this results in a generation of 1553 kW. During operation, the external electricity flow (40) has now decreased by 23% to 1237 kW.

Example 2

| | Feed (1) | Add. Feed (2) | Inlet 50″ | Outlet 50″ | Inlet 10 | Outlet 10 | Inlet flash | Outlet flash | Outlet comp |
|---|---|---|---|---|---|---|---|---|---|
| T [°C] | 380 | 250 | 304 | 450 | 419 | 950 | 40 | 40 | 218 |
| P [barg] | 28.4 | 29.4 | 28.4 | 27.4 | 26.4 | 25.0 | 23.0 | 23.0 | 88.3 |
| Components [Nm³/h] | | | | | | | | | |
| Carbon Dioxide | 247 | 0 | 247 | 247 | 263 | 158 | 158 | 158 | 158 |
| Nitrogen | 5 | 0 | 5 | 5 | 5 | 5 | 5 | 5 | 5 |
| Methane | 741 | 0 | 741 | 741 | 738 | 126 | 126 | 126 | 126 |
| Ethane | 7 | 0 | 7 | 7 | 0 | 0 | 0 | 0 | 0 |
| Hydrogen | 16 | 0 | 16 | 16 | 78 | 1808 | 1808 | 1808 | 1808 |
| Carbon Monoxide | 0 | 0 | 0 | 0 | 2 | 718 | 718 | 718 | 718 |
| Water | 0 | 1133 | 1133 | 1133 | 110 0 | 593 | 593 | 9 | 9 |
| Oxygen | 0 | 0 | 0 | 0 | 0 | 0 | 0 | 0 | 0 |
| Methanol | 0 | 0 | 0 | 0 | 0 | 0 | 0 | 0 | 0 |

Example 2 Continued

| | Add rec. | Inlet MeOH | Outlet MeOH | Inlet flash | Off-gas (22) | Recycle | Product (21) |
|---|---|---|---|---|---|---|---|
| T [°C] | 186 | 220 | 250 | 40 | 40 | 40 | 40 |
| P [barg] | 88.3 | 87.8 | 87.3 | 86.3 | 86.3 | 86.3 | 86.3 |
| Components [Nm³/h] | | | | | | | |
| Carbon Dioxide | 237 | 237 | 230 | 230 | 119 | 80 | 31 |
| Nitrogen | 8 | 8 | 8 | 8 | 5 | 3 | 0 |
| Methane | 207 | 207 | 207 | 207 | 121 | 80 | 6 |
| Ethane | 0 | 0 | 0 | 0 | 0 | 0 | 0 |
| Hydrogen | 2175 | 2175 | 924 | 924 | 551 | 367 | 6 |
| Carbon Monoxide | 786 | 786 | 171 | 171 | 101 | 67 | 2 |
| Water | 9 | 9 | 16 | 16 | 0 | 0 | 16 |
| Oxygen | 0 | 0 | 0 | 0 | 0 | 0 | 0 |
| Methanol | 3 | 3 | 625 | 625 | 4 | 3 | 618 |

### Example 3

In another example, consider the same plant and process as presented in Example 1 with the same flow of feedstock and similar operation of the e-SMR. However, in this case the plant has switched to another plant operation mode B wherein les external electricity flow (40) is available. This case is summarized in Table 3. In this case the hydrocarbon feedstock to the plant is divided in to a Feed (1) and a Fuel gas constituting respectively 73% and 27% of the full feedstock as used in Example 1. At the reduced load on the plant and when operating at an energy efficiency of 90%, the e-SMR uses 2037 kW as first electricity flow (31) in the given example. In addition the ratio between the recycle and the off-gas is switched, now sending 60% of the gas to the power generator (30), instead of 20% in Example 1. Consequently, the size of the off-gas stream increases compared to Example 1. The Fuel gas has a heating value of 6388 kcal/Nm³ while the off-gas has a heating value of 3090 kcal/Nm³, using the same electricity conversion efficiency of 48%, this results in a combined generation of 2111 kW. Consequently, the first electricity flow is fully covered by the electricity generation from the fuel streams.

Example 3

| | Feed (1) | Add. Feed (2) | Inlet 50″ | Outlet 50″ | Inlet 10 | Outlet 10 | Inlet flash | Outlet flash | Outlet comp |
|---|---|---|---|---|---|---|---|---|---|
| T [°C] | 380 | 250 | 304 | 450 | 419 | 950 | 40 | 40 | 218 |
| P [barg] | 28.4 | 29.4 | 28.4 | 27.4 | 26.4 | 25.0 | 23.0 | 23.0 | 88.3 |
| Components [Nm³/h] | | | | | | | | | |
| Carbon Dioxide | 180 | 0 | 180 | 180 | 192 | 115 | 115 | 115 | 115 |
| Nitrogen | 4 | 0 | 4 | 4 | 4 | 4 | 4 | 4 | 4 |
| Methane | 541 | 0 | 541 | 541 | 539 | 92 | 92 | 92 | 92 |
| Ethane | 5 | 0 | 5 | 5 | 0 | 0 | 0 | 0 | 0 |
| Hydrogen | 12 | 0 | 12 | 12 | 57 | 1320 | 1320 | 1320 | 1320 |
| Carbon Monoxide | 0 | 0 | 0 | 0 | 1 | 524 | 524 | 524 | 524 |
| Water | 0 | 827 | 827 | 827 | 803 | 433 | 433 | 6 | 6 |
| Oxygen | 0 | 0 | 0 | 0 | 0 | 0 | 0 | 0 | 0 |
| Methanol | 0 | 0 | 0 | 0 | 0 | 0 | 0 | 0 | 0 |

Example 3 Continued

| | Add rec. | Inlet MeOH | Outlet MeOH | Inlet flash | Off-gas (22) | Recycle | Product (21) | Fuel gas |
|---|---|---|---|---|---|---|---|---|
| T [°C] | 186 | 220 | 250 | 40 | 40 | 40 | 40 | 50 |
| P [barg] | 88.3 | 87.8 | 87.3 | 86.3 | 86.3 | 86.3 | 86.3 | 29.4 |
| Components [Nm³/h] | | | | | | | | |
| Carbon Dioxide | 173 | 173 | 168 | 168 | 87 | 58 | 23 | 67 |
| Nitrogen | 6 | 6 | 6 | 6 | 4 | 2 | 0 | 1 |
| Methane | 151 | 151 | 151 | 151 | 88 | 59 | 4 | 200 |
| Ethane | 0 | 0 | 0 | 0 | 0 | 0 | 0 | 2 |
| Hydrogen | 1588 | 1588 | 675 | 675 | 402 | 268 | 4 | 4 |
| Carbon Monoxide | 574 | 574 | 125 | 125 | 74 | 49 | 2 | 0 |
| Water | 6 | 6 | 12 | 12 | 0 | 0 | 12 | 0 |
| Oxygen | 0 | 0 | 0 | 0 | 0 | 0 | 0 | 0 |
| Methanol | 2 | 2 | 456 | 456 | 3 | 2 | 451 | 0 |

1. A plant comprising:
a first feed comprising hydrocarbons,
one or more co-reactant feeds,
an electrical steam methane reforming (e-SMR) reactor, wherein the e-SMR reactor is arranged to be heated by a first electricity flow, and wherein the e-SMR reactor is arranged to receive at least a portion of said first feed comprising hydrocarbons and at least a portion of said one or more co-reactant feeds, and generate a first syngas stream,
an upgrading section arranged to receive a syngas stream and generate at least a first product stream and an off-gas stream from said syngas stream,
a power generator arranged to receive at least a portion of said off-gas stream and/or a portion of said first feed and/or a portion of said first product stream from the upgrading section and generate a second electricity flow,
wherein at least a portion of said second electricity flow is arranged to provide at least a part of the first electricity flow to the e-SMR reactor.

2. The plant according to claim 1, comprising an external electricity flow arranged to provide part of the first electricity flow to the e-SMR reactor.

3. The plant according to claim 2, wherein a source of renewable electricity is arranged to provide said external electricity flow.

4. The plant according to claim 1, wherein the second electricity flow constitutes the entire first electricity flow required to heat the e-SMR reactor.

5. The plant according to claim 1, wherein the second electricity flow generated by the power generator is larger than the first electricity flow.

6. The plant according to claim 1, wherein the power generator is arranged to receive at least a portion of said off-gas stream and a portion of said first feed and generate a second electricity flow.

7. The plant according to claim 1, wherein said plant further comprises at least one water gas shift (WGS) reactor arranged downstream said e-SMR reactor, wherein the at least one WGS reactor is arranged to receive at least a portion of the first syngas stream from the e-SMR reactor and generate a second syngas stream from said first syngas stream, and wherein at least a portion of said second syngas stream is fed to said upgrading section.

8. The plant according to claim 1, wherein said plant further comprises one or more gas conditioning units arranged between said e-SMR reactor and said upgrading section, said one or more gas conditioning units being selected from: a flash separation unit, a CO₂ removal section, a methanator, or a combination of such units.

9. The plant according to claim 1, comprising a pre-treatment section upstream the e-SMR reactor arranged to pre-treat the first feed of hydrocarbons in combination with one or more of the co-reactant feeds before it is fed to the e-SMR reactor, wherein said pre-treatment section comprises one or more pre-treatment units selected from a gas adjustment unit, a heating unit, a hydrodesulfurisation (HDS) unit and a pre-reforming unit.

10. The plant according to claim 9, wherein a portion of the off-gas stream from the upgrading section is arranged to be returned to the pre-treatment section and used as fuel for said heating unit.

11. The plant according to claim 1, wherein first feed comprising hydrocarbons and at least a portion of said one or more co-reactant feeds are arranged to be mixed, and the e-SMR reactor is arranged to receive the mixed feed of first feed comprising hydrocarbons and one or more co-reactant feeds.

12. The plant according to claim 1, wherein the co-reactant feeds are selected from a steam feed, a hydrogen feed, or a CO₂ feed.

13. The plant according to claim 1, further comprising an electricity supply unit arranged to receive the second electricity flow from the power generator, and optionally the external electricity flow, and provide the first electricity flow to the e-SMR reactor.

14. The plant according to claim 1, wherein the upgrading section is a hydrogen purification section, a methanol synthesis section, a CO cold box, an ammonia loop, or a Fischer-Tropsch section.

15. The plant according to claim 1, wherein:
the upgrading section is a hydrogen purification section,
the first product stream is a hydrogen-rich stream, and
the off-gas stream is an off-gas stream from the hydrogen purification section.

16. The plant according to claim 1, wherein:
the upgrading section is a methanol synthesis section,
the first product stream is a methanol-rich stream, and
the off-gas stream is an off-gas stream from methanol synthesis section.

17. The plant according to claim 1, wherein:
the upgrading section is a CO cold box, and
the upgrading section being arranged to receive a syngas stream and generate a first product stream being a substantially pure CO stream, a second product stream, being a substantially pure H₂ stream and an off-gas stream from the CO cold box.

18. The plant according to claim 1, wherein:
the upgrading section is an ammonia loop,
the product stream is a substantially pure ammonia stream, and
the off-gas stream is an off-gas stream from the ammonia loop.

19. The plant according to claim 1, wherein:
the upgrading section is a Fischer-Tropsch section,
the product stream is a stream of higher hydrocarbons, and
the off-gas stream is an off-gas stream from the Fischer-Tropsch section.

20. A method for providing a product stream from a first feed comprising hydrocarbons, said method comprising the steps of:
providing a plant according to claim 1,
feeding at least a portion of the first feed comprising hydrocarbons and one or more co-reactant feeds to the electrical steam methane reforming (e-SMR) reactor, and heating said e-SMR reactor with a first electricity flow so as to generate a syngas stream from said first feed,
feeding syngas stream to the upgrading section and generating at least a product stream and an off-gas stream from said syngas stream,
feeding at least a portion of said off-gas stream and/or a portion of said first product stream from the upgrading section and/or a portion of said first feed to the power generator and generating a second electricity flow, and
feeding at least a portion of said second electricity flow as at least a part of the first electricity flow to the e-SMR reactor.

21. A method for operating a plant according to claim 1, wherein;
in a first plant operation mode A, the first electricity flow to the e-SMR reactor, comprises a first proportion (A1) of the second electricity flow and a first proportion (A2) of the external electricity flow;
in a second plant operation mode B, the first electricity flow to the e-SMR reactor, comprises a second proportion (B1) of the second electricity flow and a second proportion (B2) of the external electricity flow;
wherein the first proportion (A1) of the second electricity flow in the first plant operation mode A is smaller than the second proportion (B1) of the second electricity flow in the second plant operation mode B;
and wherein the first proportion (A2) of the external electricity flow in the first plant operation mode A is larger than the second proportion (B2) of the external electricity flow in the second plant operation mode B; and
said method comprising the step of switching from plant operation mode A to plant operation mode B or vice-versa.

22. The method according to claim 21, wherein the first electricity flow in the second plant operation mode B is lower than the first electricity flow in the first plant operation mode A.

23. The method according to claim 21, wherein — in the first plant operation mode A - the first proportion (A1) of the second electricity flow in the first electricity flow is 50% or less, 30% or less, 10% or less, or 0%.

24. The method according to claim 21, wherein — in the second plant operation mode B - the second proportion (B1) of the second electricity flow in the first electricity flow is 75% or more, 80% or more, 90% or more, or 100%.

25. The method according to claim 21, wherein; in the second plant operation mode B, the first electricity flow to the e-SMR reactor consists of the second electricity flow; and the second proportion (B2) of the external electricity flow is zero.

26. The method according to claim 21, wherein the step of switching from plant operation mode A to plant operation mode B is at least partially obtained by increasing off-gas production in the upgrading section.

27. The method according to claim 21, wherein the step of switching from plant operation mode A to plant operation mode B is at least partially obtained by feeding part of said first feed directly to said power generator .

28. The method according to claim 21, wherein the step of switching from plant operation mode A to plant operation mode B is at least partially obtained by decreasing said first electricity flow.

29. The method according to claim 21, wherein said external electricity flow is provided from a renewable source of electricity, and wherein said step of switching from plant operation mode A to plant operation mode B takes place when the external electricity flow available from said renewable source of electricity drops below a predetermined level.

30. The method according to claim 21, wherein said external electricity flow is provided from a renewable source of electricity, and wherein said step of switching from plant operation mode B to plant operation mode A takes place when the external electricity flow available from said renewable source of electricity rises above a predetermined level.

31. The method according to claim 21, wherein said switch between operation mode A and B, or vice versa, takes place within a time period of 2 hours after a preceding switch.
